(19) United States
(12) Patent Application Publication
(10) Pub. No.: US 2018/0235489 A1
(43) Pub. Date: Aug. 23, 2018

(54) PHOTOPLETHYSMOGRAPHIC WEARABLE BLOOD PRESSURE MONITORING SYSTEM AND METHODS

(71) Applicant: SENSOGRAM TECHNOLOGIES, INC., Plano, TX (US)

(72) Inventors: Vahram Mouradian, Plano, TX (US); Armen Poghosyan, Plano, TX (US)

(21) Appl. No.: 15/897,876

(22) Filed: Feb. 15, 2018

Related U.S. Application Data
(60) Provisional application No. 62/460,504, filed on Feb. 17, 2017.

(51) Int. Cl. A61B 5/024; A61B 5/00; A61B 5/021

(52) U.S. Cl. CPC A61B 5/02416; A61B 5/0205; A61B 5/02141; A61B 5/681

(57) ABSTRACT

A photoplethysmographic (“PPG”) device having a housing, a band and a first optical sensor. The band extends from the housing and is configured to enclose an attachment site on a user's body. The PPG device has an inner surface oriented to face the attachment site and an outer surface oriented to face away from the attachment site. The first optical sensor includes at least one first optical emitter oriented to selectively emit a respective first light from the outer surface, and at least one first optical receiver oriented to receive a reflected portion of the respective first light returning to the outer surface. The respective first light is selected from one or more wavelengths operative to reflect from one or more of oxyhemoglobin and deoxyhemoglobin at an observation site on the user's body, the observation site being remote from the attachment site.

This application claims priority to U.S. provisional patent application No. 62/460,504 filed on Feb. 17, 2017, which is incorporated herein by reference.

### RELATED APPLICATIONS

This application is related to: U.S. application Ser. No. 15/365,242 filed on Nov. 30, 2016; U.S. application Ser. No. 14/674,499 filed on Mar. 31, 2015; U.S. provisional patent application No. 61/972,905 filed on Mar. 31, 2014; U.S. application Ser. No. 14/675,639 filed on Mar. 31, 2015; and U.S. provisional patent application No. 61/973,035 filed on Mar. 31, 2014, the disclosures of which are incorporated herein by reference for all purposes.

### TECHNICAL FIELD

The invention relates in general to photoplethysmographic (“PPG”) measurement systems and apparatus using optical sensors, and in particular to non-invasive human blood pressure measurements by wearable optical-sensing systems.

### BACKGROUND OF THE INVENTION

Blood pressure measurement techniques are generally classified as either instant or continuous. Instant blood pressure measurement means checking blood pressure at certain points in time, like a spot check, while continuous measurement is checking a patient's blood pressure constantly, with every heartbeat. Instant techniques of blood pressure measurement involve some kind of sensor working for a short period of time, such as sphygmomanometer cuffs that operate non-invasively. The disadvantage of such techniques is that they can miss intermittent blood pressure changes, and the administration of the measurement can be cumbersome, difficult, and uncomfortable. This is particularly true where the subject is under intensive care or is in an assisted living environment (e.g., the elderly), in which blood pressure measurements are taken frequently. Continuous blood pressure measurement techniques typically employ an invasive device such as an arterial catheter, from which instant blood pressure can be tracked in real time. Continuous blood pressure measurement devices have the disadvantage of being invasive to the body, and often are not amenable to use outside controlled environments, such as the surgical theater.

Photoplethysmography or photoplethysmographic (PPG) systems have been used in an attempt to measure various physiological characteristics including, but not limited to, the blood-oxygen saturation of hemoglobin in arterial blood, the volume of individual blood pulsations supplying the tissue, and the rate of blood pulsations corresponding to each heartbeat of a patient. Attempts at measuring some of these characteristics have used a non-invasive PPG sensor, which scatters light through a portion of the patient's tissue where blood is perfused through the blood vessels (capillaries and arteries) and optically senses the absorption and/or reflection of light in such tissue.

Typical PPG measurement systems include an optical sensor worn on the tip of a patient's appendage (e.g., a finger, an earlobe, etc.). The sensor has a photoemitter that directs light signals into the appendage where the sensor is attached, and a photoreceiver that detects light reflected by or transmitted through the tissue. In the reflection mode, some portion of light is absorbed and the remaining portion is reflected back to the photoreceiver. In the transmission mode, some portion of light is absorbed, and the remaining portion is transmitted through the tissue to the photoreceiver. The operation mode depends on the configuration and intended use of the optical sensor. For example, fingertip sensors are often configured to operate in the reflection mode, whereas earlobe sensors are often configured to operate in transmission mode. The intensity of the light received by the photoreceiver is monitored to provide one or more intensity signals, which can be resolved into a waveform indicating relative values of blood flow rate at the measured location. These intensity signals are used to compute blood parameters, but the waveform produced by the signal does not directly indicate blood pressure.

PPG measurement systems have been provided in various operative system configurations. For example, it is known to provide optical sensors that are electrically connected to separate computing and display systems for use in operative or in-patient care situations. It is also known to provide wearable PPG sensor systems that include PPG sensors and data collection systems in a self-contained configuration that can be worn on the patient's body, such as the wrist. Such systems may include wireless communication capabilities (e.g., by Bluetooth technology or the like), communication ports for wired communications and data transfer, and the like.

There remains a need to provide alternative techniques and systems for measuring blood pressure.

### SUMMARY

In a first aspect, there is provided a photoplethysmographic (“PPG”) device having a housing, a band, and a first optical sensor. The band extends from the housing and is configured to enclose an attachment site on a user's body. One or both of the band and the housing having an inner surface oriented to face the attachment site when the PPG device is mounted at the attachment site and an outer surface oriented to face away from the attachment site when the PPG device is mounted at the attachment site. The first optical sensor includes at least one first optical emitter oriented to selectively emit a respective first light from the outer surface, and at least one first optical receiver oriented to receive a reflected portion of the respective first light returning to the outer surface. The respective first light is selected from one or more wavelengths operative to reflect from one or more of oxyhemoglobin and deoxyhemoglobin at an observation site on the user's body. The observation site is remote from the attachment site.

The housing and band may be integrated into a single structure.

The attachment site may be a human wrist, and the band may be shaped and sized to attach to the human wrist.

The at least one first optical emitter may include two first optical emitters. The two first optical emitters may include a first light-emitting diode (“LED”) configured to emit a respective first light including red light at a wavelength of about 580 to 660 nanometers, and a second LED configured to emit a respective first light including infrared light at a wavelength of about 880 to 940 nm nanometers.

The observation site may be a human finger, and the first optical sensor may be configured to emit the light and receive the reflected portion of the light from the human finger. The first optical sensor may be located in a depression or surrounded by a shroud.

The PPG device may include a display unit configured to indicate at least one of: a blood pressure measurement; a pulse measurement; a respiration rate measurement; or a blood oxygen level measurement.

The housing and the band may be configured as an attachment to a watch or a smartwatch.

The PPG may also include a second optical sensor having at least one second optical emitter oriented to selectively emit a respective second light from the inner surface, and at least one second optical receiver oriented to receive a reflected portion of the respective second light returning to the inner surface. The respective second light is selected from one or more wavelengths operative to reflect from one or more of oxyhemoglobin and deoxyhemoglobin at the attachment site. The at least one second optical emitter may include two second optical emitters. The two second optical emitters may include a third LED configured to emit a respective second light including red light at a wavelength of about 580 to 660 nanometers, and a fourth LED configured to emit a respective second light including infrared light at a wavelength of about 880 to 940 nm nanometers.

The PPG device may also include a first electrode contact positioned on the outer surface and oriented to be selectively contacted by the observation site, a second electrode contact positioned on the inner surface an oriented to contact the attachment site, and a circuit operatively connected to the first electrode contact and the second electrode contact, the circuit being configured to derive one or more physiological conditions of the user when the user contacts the first electrode contact with the observation site. The circuit may be a galvanic skin response circuit. The circuit may be configured to detect electrical impulses generated by the user's muscles.

### BRIEF DESCRIPTION OF THE DRAWINGS

Embodiments of the invention will now be described, strictly by way of example, with reference to the accompanying drawings, in which:

FIG. 1 is a schematic representation of a first exemplary PPG device.

FIG. 2 is a schematic representation of a second exemplary PPG device.

FIG. 3 is a schematic representation of a third exemplary PPG device.

### DESCRIPTION OF THE EMBODIMENTS

It has been found that typical wearable PPG systems can be somewhat ineffective or cumbersome. For example, wearable systems that include a fingertip optical sensor are cumbersome to the wearer. The optical sensor interferes with digital manipulation, making certain everyday hand movements (e.g. driving, eating, etc.) difficult or impossible, and may require an electrical harness to connect the fingertip sensor to the rest of the wearable system. As another example, a wrist-mounted PPG system may include an optical sensor system having an optical emitter that directs light into the wearer's wrist region, and an optical receiver that senses light reflected from the wrist region. While such devices can be no more cumbersome than a wristwatch, the wrist is not an ideal portion of the body for PPG analysis because it lacks the large number of detectable capillaries present in other parts of the body, such as the fingertips. Thus, wrist-mounted PPG devices can suffer from reduced accuracy or consistency.

Embodiments of the invention provide improved or alternative utility for wrist-based and other wearable PPG devices. For example, the invention may be a wearable PPG device having an outward-facing optical sensor, rather than a conventional inward-facing optical sensor. As another example, the invention may be a wearable PPG device having an outward-facing optical sensor and an inward-facing optical sensor. Non-limiting examples of the invention are described herein in detail, with the understanding that other embodiments and alterations and variations to the shown embodiments may be made while still remaining within the spirit and scope of the invention.

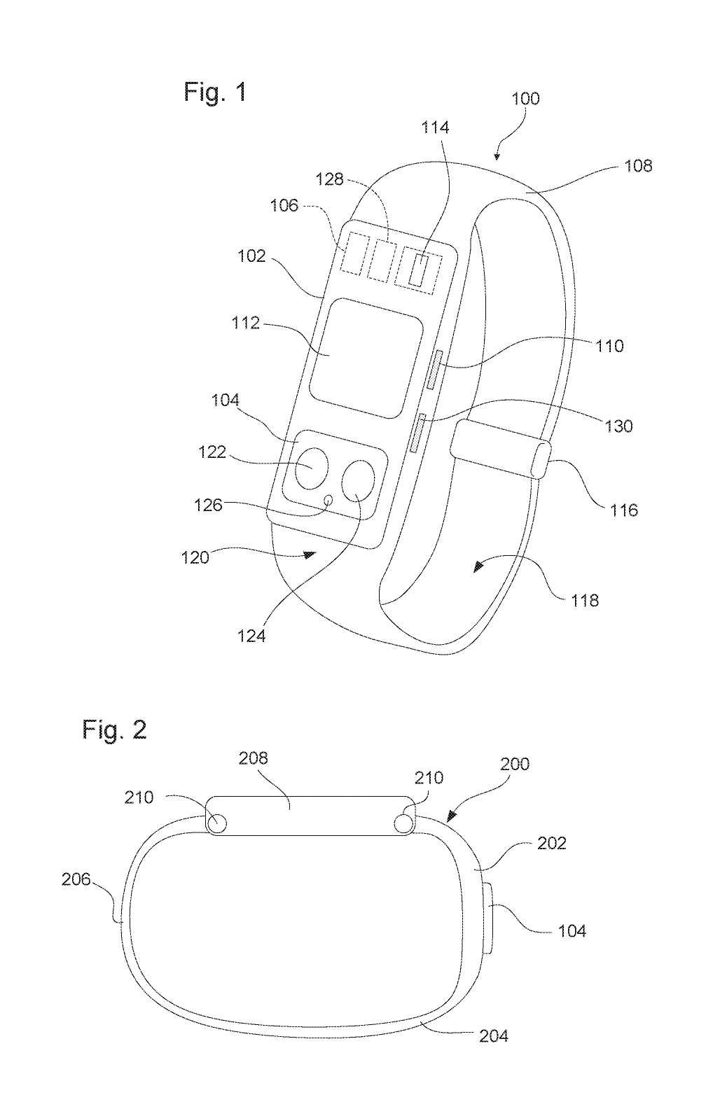

FIG. 1 illustrates a first exemplary embodiment of a PPG device 100. The PPG device 100 includes a housing 102, an optical sensor 104 mounted to the housing 102, a processing unit 106 mounted to the housing 102, and a band 108, which may be mounted to or formed integrally with the housing 102. The PPG device 100 also may include a control unit 110, a display unit 112, a communication unit 114, and other features such as described below.

The housing 102 provides a shell or platform to which the remaining parts are directly or indirectly attached. A plastic or metallic structure is expected to be suitable for most embodiments. For example, the housing 102 may be injection-molded plastic, cast magnesium, machined aluminum or steel, or the like. The housing 102 may include surface coatings or other features such as a water-resistant shell, a glass or transparent polycarbonate face, or the like. Other alternatives will be apparent to persons of ordinary skill in the art in view of the present disclosure.

The housing 102 and band 108 preferably are configured with shapes and dimensions similar to a conventional wristwatch or smart watch. The housing 102 and band 108 also may comprise a conventional wristwatch or smart watch to which additional features such as discussed below are added to form an embodiment of the invention. In one example, the housing 102 may have a generally flat rectangular or rounded shape that extends in a plane with a maximum dimension in the plane of approximately two inches or less, and a thickness extending perpendicular to the plane of approximately one-half inch or less. The band 108 may be attached to edges of the housing 102 and configured to encircle a volume having a diameter of about two to three inches, or such a size as corresponds to the typical dimensions of a human wrist. The band 108 may have an openable portion with a clasp 116 or the like for selective attachment and detachment of the band 108 to the wearer, or it may be flexible enough to stretch over the wearer's hand to be placed on the wrist. The housing 102 and band 108 optionally may be provided with conventional wristwatch features, such as a bezel, face and mechanical movement or digital clock for telling time.

The PPG device 100 has an inner surface 118 that faces the wearer, and an outer surface 120 that faces away from the wearer. The inner and outer surfaces 118, 120 may be formed by the housing 102, band 108 or both. For example, where the band 108 is connected along edges of the housing 102, the band 108 and housing 102 may collectively form the inner and outer surfaces 118, 120. As another example, the housing 102 may be mounted to the surface of a continuous band 108, such that only the band 108 forms the inner surface 118 while the band 108 and housing 102 together form the outer surface 120.

As discussed herein, the housing 102 and band 108 are described as being configured for mounting on the human wrist. However, in other embodiments, the housing 102 and band 108 may have different shapes or sizes. For example, the band 108 may be dimensioned to wrap around an upper arm, a leg, an ankle, a torso, or the like. Use in non-human subjects is also contemplated.

The optical sensor 104 is a non-invasive device for evaluating vital signs, such as heart rate, respiration rate, oxygen saturation and blood pressure. For example, the optical sensor 104 may be configured as a pulse oximeter system that uses light to detect different absorption levels of oxyhemoglobin and deoxyhemoglobin in blood in the underlying tissue. By measuring the difference in absorbance at various wavelengths, the degree of blood oxygen saturation can be estimated. Heart rate also may be determined by observing the period of fluctuations in received light pulses. Blood pressure measurement also may be determined, and this and other uses for optical sensors 104 are disclosed in U.S. application Ser. No. 15/365,242, which is incorporated herein by reference. Additional uses also may be made of the optical sensor 104, as known in the art.

The optical sensor 104 is oriented to face away from the portion of the body to which the PPG device 100 is attached. For example, the optical sensor 104 may comprise one or more optical emitters 122 oriented to emit light from the outer surface 120, and one or more optical receivers 124 that are oriented to receive light striking the outer surface 120.

The optical emitters 122 may be selected to transmit one or more particular wavelengths of light into the user's tissue. The amount of such wavelengths that are reflected back to the optical detectors 124 can be evaluated to determine vital information such as pulse rate and blood oxygen content, as known in the art. For example, in certain embodiments, the optical emitter 122 may have a red light-emitting diode (“LED”) that transmits red light at a wavelength of about 580 to 660 nm, and an infrared LED that emits infrared light at a wavelength of about 880 to 940 nm. The two LEDs may be controlled by a processing unit 106, which may selectively activate the two LEDs by controlling their respective current management schemes. Control systems for optical emitters 122 such as described above are generally known in the art and require no further explanation herein.

Similarly, the optical receivers 124 may comprise one or more photodiodes configured to detect light at particular wavelengths. For example, one photodiode may detect light at a wavelength of about 580 to 660 nm, and a second photodiode may detect light at a wavelength of about 880 to 940 nm. Other embodiments may use a single photodiode, and the light of different wavelengths, such as red and infrared, may be time multiplexed to differentiate between light emitted from the two different LEDs. The signal detected by a single photodiode may be demultiplexed to extract the two different light signals. The demultiplexing frequency preferably is much high higher than the blood pulse rate, to provide sufficient resolution for accurately evaluating the received signal. Such devices and their operation and control are known in the art and examples are described in applications to which this application claims priority, and need not be described further herein.

In operation, the optical receivers 124 generate respective signals corresponding to the intensity of the light that reaches the optical receivers 124. To this end, the optical receivers 124 may comprise photodiodes or the like. Photodiodes generate a current that is proportional to the intensity of the received light—the greater the light received, the greater the current generated by the photodiode.

The optical sensor 104 is operatively connected to the processing unit 106, which is configured to operate and control the optical sensor 104 and other systems necessary to perform PPG measurements. The processing unit 106 may include various known signal processing features, such as amplifiers, a DC subtraction circuit, an analog-to-digital converter, a noise reduction circuit, and so on. The processing unit 106 also may include a memory for storing data, firmware instruction, and the like. The processing unit 106 also may include, or be operatively connected to, one or more communication units 114, such as wireless communication devices (e.g., Bluetooth, Zigbee or near field communication transceivers, mobile telephony (e.g., GSM) devices, etc.) or wired communication devices (e.g., a micro-USB or USB-to-UART (“Universal Asynchronous Receiver and Transmitter”) port, or the like).

The processing unit 106 may include any suitable microprocessor or collection of microprocessors or electronic components. An ultra-low power microprocessor is preferred. In one embodiment, the processing unit 106 may be based on the 32bit ARM Cortex-M4 core, which includes a variety of peripheral devices. The microprocessor may have an ultra-low power consumption of about 238 pA/MHz in dynamic run mode, and 0.35 pA in lowest power mode. The processing unit 106 preferably has sufficient power and speed to allow essentially continuous collection and processing of data from the optical sensor 104, but it may be configured to alternate between data collection and data processing stages to conserve power consumption.

The details of processing units 106 and peripheral devices that can be used in a PPG device 100 are generally known in the art, and need not be described in detail herein.

During operation, the processing unit 106 initiates operation of the optical sensor 104 to begin data collection. The optical sensor 104 operates by emitting light from the one or more optical emitters 122 and detecting the quantity of reflected light at the one or more optical receivers 124. Data from the optical receivers 124 can be referred to generally as the “PPG signal.” The PPG signal may be collected, stored and transmitted in a variety of forms, such as raw analog current values, partially-processed conditioned signals, graphical PPG waveform representations, and so on. The PPG signal may be used for a number of purposes, as will be appreciated by persons of ordinary skill in the art.

The processing unit 106 may collect the output of the optical receivers 124, processes the data, and provides an output of PPG signal. For example, the processing unit 106 may include a program application to process the PPG signal to estimate certain physical conditions of the user, such as the user's pulse, blood oxygen saturation, respiration rate, blood pressure, and other vital signs or physiological properties. As an alternative, the processing unit 106 may send the raw or partially-processed data from the optical receivers 124 to an external system for further processing. For example, the processing unit 106 may perform amplification, and analog-to-digital conversion on the optical receivers' 124 output signals, and send this as partially-processed data to a smartphone or other computer that is connected wirelessly or by a wired connection to the PPG device 100. The other device then continues processing the data to provide the desired output information. As another example, the processing unit 106 may directly transmit the output of the optical receivers 124 to an external device, such as a smartphone or other computer, which then takes the data and converts it into the final desired format. The processing unit 106 also may store the partially-processed data in a memory until such time that it is transmitted to the other device.

One or more batteries 128, such as a rechargeable lithium-ion battery pack, may be provided in the PPG device 100 and operatively connected to the features to provide power. A charging port 130 (which may be combined with a wired communication port, such as in a conventional powered USB device) may be provided for recharging the batteries 128, but a wireless induction recharging circuit also may be used. The processing unit 106 or other processors may be configured to control power distribution and management, as known in the art.

As noted above, the PPG device 100 also may include a control unit 110. The control unit 110 provides a user interface to operate one or more functions of the PPG device 100. For example, the control unit 110 may include a power switch to turn the PPG device 100 (or particular systems thereof) on an off, a mode switch to change the operating mode of the PPG device 100 (e.g., between clock mode and PPG data collection mode), and one or more toggle and selection switches to enable scrolling through and selecting menu options. The various switches may be multifunction switches with mode-sensitive functionality that changes depending on the particular mode in which the PPG device 100 is operating, and so on.

The display unit 112 provides a visual output for the user, and may comprise a liquid crystal display screen, one or more LEDs, a full-color display screen, or the like. In one embodiment, the display unit 112 provides a visual indication of vital signs, such as blood pressure, blood oxygen content, heart rate, respiration rate, and so on. Such information may be produced internally within the PPG device 100, or it may be retransmitted back to the PPG device 100 after the raw or partially-processed PPG signal is processed at a remote computer. For example, the PPG device 100 may collect the PPG signal, send it to a smartphone or remote computer for processing, and then receive the processed information back at the PPG device 100 for display on the display unit 112. The display unit 112 also may be provided at a separate physical location than the remainder of the PPG device 100. For example, the display unit 112 may be integrated into a program application operating on a smartphone or computer to display the vital information there. It will also be appreciated that the control unit 110 may be integrated into the display unit 112. For example, the integrated control and display unit may comprise an interactive touchscreen or the like. Similarly, the optical sensor 104 also may be integrated into the display unit 112. Other alternatives will be apparent to persons of ordinary skill in the art in view of the present disclosure.

In use, the user contacts the optical sensor 104 to a part of the user's body to perform PPG data collection. The optical sensor 104 faces away from the portion of the body to which the PPG device 100 is attached, so some other portion of the body must be brought into view of the optical sensor 104 for proper data collection. For clarity, the portion of the body to which the PPG device 100 is attached by the band 108 is referred to as the attachment site, and the portion of the body that is observed by the optical sensor is referred to as the observation site.

The observation site preferably is a fingertip or other region having a high density of capillaries, which can help ensure the most accurate readings. The optical sensor 104 may be shaped to obtain favorable measurements from the finger by positioning the optical emitters 122 and optical receivers 124 close together (e.g., within a span of a typical fingertip—a distance of about 0.5 inches or less or the like). The optical sensor also may be placed in a recess within the surface of the housing 102 (or include a shroud surrounding the optical emitters 122 and optical receivers 124) to help prevent stray light from striking the observation site during measurement. Such a depression or shroud also may provide a good tactile indicator when the user has properly placed the fingertip above the optical sensor 104. If it is found that a shroud or depression is not necessary to facilitate more accurate readings, it still may be desirable to place one or more tactile indicators (e.g., bumps or ridges) at or near the optical sensor 104 to help assist with proper finger placement, or a graphic image may be placed on the housing 102 at the proper location to help guide the user.

It is desirable from an energy conservation standpoint to activate the operation of the optical emitters 122 and optical receivers 124 only when it is desired to perform PPG data collection. To this end, the PPG device 100 may be configured to activate the optical emitters 122 and optical receivers 124 at predetermined times or upon manual initiation of PPG data collection. For example, the PPG device 100 may include an alarm that periodically alerts the wearer that PPG data collection is necessary, at which point a countdown is initiated to give the wearer time to place the observation site against the optical sensor 104. The PPG device 100 also may include an activation sensor 126, such as a photodetector of contact sensor, that detects when the observation site is located adjacent the optical sensor 104. The PPG device 100 also may be programmed to initiate PPG data collection upon the wearer's request, such as by providing a menu option to begin testing, or providing automatic test initiation upon holding the observation site in continuous contact with the activation sensor 126 for a predetermined amount of time. Other options will be readily apparent in view of the present disclosure.

Once the observation site is in position next to or against the optical sensor 104, the optical emitter 122 can transmit light into the user's tissue, and the optical receiver 124 can receive light reflected from the blood within the tissue to perform photoplethysmographic measurements. The display unit 112 may provide a visual signal (e.g., a timer or “complete” signal) and/or an audible signal to inform the user that PPG data collection is proceeding or complete. The PPG device 100 also may signal when there is an error in the PPG data collection, such as a misplacement of the observation site or poor data collection, so give the user an opportunity to correct the process.

The foregoing embodiment is expected to provide a number of benefits over conventional PPG devices. For example, it is wearable without being unduly cumbersome, and can be configured to attach to any number of body regions. The PPG device 100 also helps address the problem of obtaining optimal PPG data when the device is mounted on a body region that may be relatively ineffective at providing PPG data, such as the wrist. The use of an outward-facing optical sensor 104 also provides a more deliberate and controlled PPG data collection process. This can conserve battery power by avoiding continuous PPG data collection, and avoid PPG data collection when the user does not desire it or when it might be inappropriate (e.g., it may not be desirable to collect data during certain activities when they might not be representative of the user's normal condition or condition during the time of interest for data collection purposes).

The PPG device 100 also may be configured to fit into or integrate with existing watch or smartwatch technologies with little or no change to those devices. For example, as shown in FIG. 2, a PPG device 200 may be configured as a housing 202 and band 204 in the form of a strap 206 to which a wristwatch or smartwatch 208 is attached. For example, the PPG device 200 may replace the original smartwatch band and be attached at the smartwatch's existing band pins 210. The PPG device 200 alternatively could be shaped to conform to or mount on the existing smartwatch band. Alternatively, the operative features may be integrated into the existing housing of a wristwatch or smartwatch. Other possible benefits and configurations will be apparent with further review and implementation of embodiments of the invention.

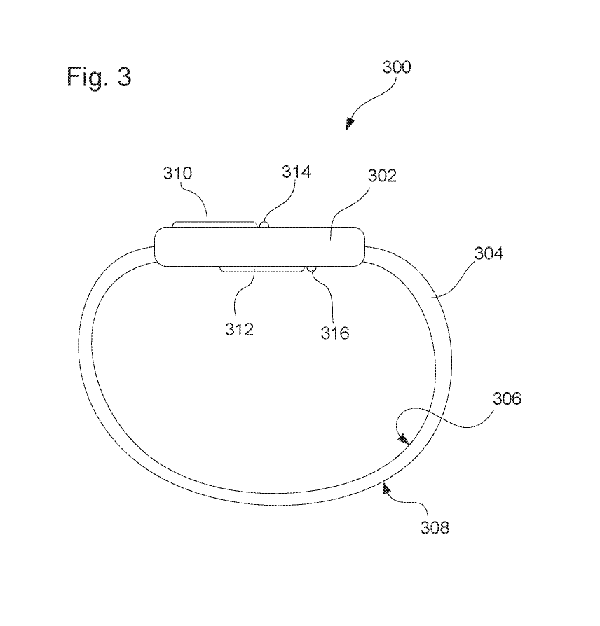

Another exemplary embodiment of a PPG device 300 is illustrated in FIG. 3. In this embodiment, the PPG device 300 comprises a housing 302, a band 304, an inner surface 306 and an outer surface 308. An outward-facing first optical sensor 310 is provided and positioned to transmit and receive light via the outer surface 308, and an inward-facing second optical sensor 312 is provided and positioned to transmit and receive light via the inner surface 306. The PPG device 300 may include other features, such as described elsewhere herein, and it is not necessary to illustrate or explain such features in detail.

The first optical sensor 310 has one or more outer optical emitters and one or more outer optical receivers, and may operate like the optical sensor 104 of the first embodiment to collect PPG data at an observation site that is remote from the attachment site to which the PPG device 300 is connected. The second optical sensor 312 includes one or more inner optical emitters and one or more inner optical receivers. The second optical sensor 312 is configured to collect PPG data from the user at the attachment site. A processor (not shown), such as described above, is provided to operate the first and second optical sensors 310, 312 to perform data collection at the desired times.

The embodiment of FIG. 3 is expected to provide additional functionality by allowing continuous or automatic PPG data collection via the second optical sensor 312, and periodic manual PPG data collection via the first optical sensor 310. A number of uses are envisioned, and a number of benefits can be obtained from this combination of functions. For example, the second optical sensor 312 may be used to periodically or continuously collect PPG data for evaluating the user's pulse rate, blood oxygen content, and/or respiration rate, and the first optical sensor 310 may be periodically used to generate relatively accurate PPG data collection when it is desired to determine blood pressure. As another example, the second optical sensor 312 may be used to continuously or periodically monitor blood pressure (and other vitals), and the user may be prompted to perform PPG data collection using the first optical sensor 310 at predetermined intervals or when information obtained from the second optical sensor 312 indicates that a more accurate measurement should be taken using the first optical sensor 310. As another example, the second optical sensor 312 may continuously monitor blood oxygen level, and the first optical sensor 310 may be used to periodically measure blood pressure or other vitals. In another example, the first optical sensor 310 is used to periodically evaluate blood pressure or other vital signs based on PPG measurements, and the second optical sensor 310 is used as a backup at such times that the user may forget to use or be unable to use the first optical sensor 310 (e.g., during sleep). In yet another example, the second optical sensor 312 comprises a single optical emitter and a single optical receiver, and is configured only to indicate pulse rate, whereas the first optical sensor 310 comprises multiple optical emitters and one or more optical receivers and is configured to evaluate blood oxygen content or blood pressure. Other alternatives will be apparent to persons of ordinary skill in the art in view of the present disclosure.

Embodiments of the invention also may include additional sensor systems for monitoring the user. For example, the PPG device 300 of FIG. 3 may include an outer electrode contact 314 on the outer surface 308 and an inner electrode contact 316 on the inner surface 306. The inner electrode contact 316 contacts the user's body at the attachment site, and the user can periodically contact the outer electrode contact 314 with an observation site, such as a finger. When the user makes contact with both electrode contacts 314, 316 the user's body completes an electric circuit. Inside the PPG device 300, the electrode contacts 314, 316 are connected to a processor (e.g., processor 106 or a separate dedicated processor) that is configured to analyze the body's electrical behavior. For example, the processor may comprise a galvanic skin response circuit that is configured to transmit an electric stimulus through the contacts 314, 316 to the skin, and measure the skin's reaction to assess variations in electrical conductance that might be representative of certain physiological conditions. The contacts 314, 316 also may be connected to a circuit that measures electrical impulses generated by the user's muscles (including the heart) to evaluate heart rate and other body activity. Operative features of electrical monitoring devices having contacts through which electrical properties of the body are detected are generally known, and need not be described in greater detail herein.

Other alternatives for functions for an integrated electrical contact system will be apparent to persons of ordinary skill in the art in view of the present disclosure. For example, the embodiment of FIG. 1 may be provided with electrode contacts 314, 316 such as described above, to provide electrical heart rate monitoring when the user contacts the outer electrode contact 314.

The present disclosure describes a number of new, useful and nonobvious features and/or combinations of features that may be used alone or together. The embodiments described herein are all exemplary, and are not intended to limit the scope of the inventions. It will be appreciated that the features shown and described in documents incorporated herein by reference may be added to embodiments in a manner corresponding to the use of such features in the incorporated references. It will also be appreciated that the inventions described herein can be modified and adapted in various ways, and all such modifications and adaptations are intended to be included in the scope of this disclosure and the appended claims.

1. A photoplethysmographic (“PPG”) device comprising:
a housing;
a band extending from the housing and configured to enclose an attachment site on a user's body, one or both of the band and the housing having an inner surface oriented to face the attachment site when the PPG device is mounted at the attachment site and an outer surface oriented to face away from the attachment site when the PPG device is mounted at the attachment site; and
a first optical sensor comprising:
at least one first optical emitter oriented to selectively emit a respective first light from the outer surface, and
at least one first optical receiver oriented to receive a reflected portion of the respective first light returning to the outer surface,
wherein the respective first light is selected from one or more wavelengths operative to reflect from one or more of oxyhemoglobin and deoxyhemoglobin at an observation site on the user's body, the observation site being remote from the attachment site.

2. The PPG device of claim 1, wherein the housing and band are integrated into a single structure.

3. The PPG device of claim 1, wherein the attachment site is a human wrist, and the band is shaped and sized to attach to the human wrist.

4. The PPG device of claim 1, wherein the at least one first optical emitter comprises two first optical emitters.

5. The PPG device of claim 4, wherein the two first optical emitters comprise:
a first light-emitting diode (“LED”) configured to emit a respective first light comprising red light at a wavelength of about 580 to 660 nanometers; and
a second LED configured to emit a respective first light comprising infrared light at a wavelength of about 880 to 940nm nanometers.

6. The PPG device of claim 1, wherein the observation site comprises a human finger, and the first optical sensor is configured to emit the light and receive the reflected portion of the light from the human finger.

7. The PPG device of claim 6, wherein the first optical sensor is located in a depression or surrounded by a shroud.

8. The PPG device of claim 1, further comprising a display unit configured to indicate at least one of: a blood pressure measurement; a pulse measurement; a respiration rate measurement; or a blood oxygen level measurement.

9. The PPG device of claim 1, wherein the housing and the band are configured as an attachment to a watch or a smartwatch.

10. The PPG device of claim 1, further comprising a second optical sensor comprising:
at least one second optical emitter oriented to selectively emit a respective second light from the inner surface; and
at least one second optical receiver oriented to receive a reflected portion of the respective second light returning to the inner surface;
wherein the respective second light is selected from one or more wavelengths operative to reflect from one or more of oxyhemoglobin and deoxyhemoglobin at the attachment site.

11. The PPG device of claim 10, wherein the at least one second optical emitter comprises two second optical emitters.

12. The PPG device of claim 11, wherein the two second optical emitters comprise:
a third LED configured to emit a respective second light comprising red light at a wavelength of about 580 to 660 nanometers; and
a fourth LED configured to emit a respective second light comprising infrared light at a wavelength of about 880 to 940nm nanometers.

13. The PPG device of claim 10, further comprising:
a first electrode contact positioned on the outer surface and oriented to be selectively contacted by the observation site;
a second electrode contact positioned on the inner surface an oriented to contact the attachment site; and
a circuit operatively connected to the first electrode contact and the second electrode contact, the circuit being configured to derive one or more physiological conditions of the user when the user contacts the first electrode contact with the observation site.

14. The PPG device of claim 13, wherein the circuit comprises a galvanic skin response circuit.

15. The PPG device of claim 13, wherein the circuit is configured to detect electrical impulses generated by the user's muscles.

16. The PPG device of claim 1, further comprising:
a first electrode contact positioned on the outer surface and oriented to be selectively contacted by the observation site;
a second electrode contact positioned on the inner surface an oriented to contact the attachment site; and
a circuit operatively connected to the first electrode contact and the second electrode contact, the circuit being configured to derive one or more physiological conditions of the user when the user contacts the first electrode contact with the observation site.

17. The PPG device of claim 16, wherein the circuit comprises a galvanic skin response circuit.

18. The PPG device of claim 16, wherein the circuit is configured to detect electrical impulses generated by the user's muscles.
